(19) United States
(12) Patent Application Publication
(10) Pub. No.: US 2017/0226523 A1
(43) Pub. Date: Aug. 10, 2017

(54) METHODS FOR SETARIA VIRIDIS TRANSFORMATION

(71) Applicant: Benson Hill Biosystems, Inc., ResearchTriangle Park, NC (US)

(72) Inventors: Xiuhua Chen, St. Louis, MO (US); Xingrong Wu, St. Charles, MO (US)

(73) Assignee: Benson Hill Biosystems, Inc., ResearchTriangle Park, NC (US)

(21) Appl. No.: 15/501,916

(22) PCT Filed: Aug. 6, 2015
(86) PCT No.: PCT/US2015/043989

§ 371 Date: Feb. 6, 2017

Related U.S. Application Data
(60) Provisional application No. 62/034,531, filed on Aug. 7, 2014.

(51) Int. Cl. C12N 15/82; A01H 4/00

(52) U.S. Cl. CPC C12N 15/8205; C12N 15/8203; A01H 4/008

(57) ABSTRACT

This invention relates to methods for the transformation of Setaria species such as Setaria viridis and transformed plants produced according to the method. Specifically, this invention relates to direct transformation of callus derived from mature embryos using Agrobacterium-mediated transformation, and plants regenerated from the transformed callus tissue. The methods comprise utilizing Setaria mature embryos as the source of plant material for callus induction; induced calli can be infected by Agrobacterium hosting an appropriate vector. Transgenic plants are regenerated from transgenic calli grown under conditions favoring growth of transformed cells while substantially inhibiting growth of non-transformed cells. These methods provide for significantly increased plant transformation efficiency with minimal ratio of escapes.

### FIELD OF THE INVENTION

The invention is drawn to plant genetic transformation, particularly to methods for the transformation of Setaria species.

### BACKGROUND OF THE INVENTION

Current protocols for S. viridis transformation use callus derived from mature embryos as the target tissue for Agrobacterium-mediated transformation. Agrobacterium-mediated transformation is performed by co-cultivation of Agrobacterium cells harboring the transformation vector with the plant tissue to be transformed. After the Agrobacterium cells are substantially removed from the plant tissue, the plant tissue is then transferred to selection medium. This selection medium contains appropriate chemicals (e.g., antibiotics and/or herbicides) to select for transformed cells. Following selection, plant tissue is transferred to regeneration medium, where shoots are produced. These growing shoots are then transferred to rooting medium. Following root development, plantlets are then transferred to soil for cultivation.

Optimizing transformation protocols for a plant species requires optimization of the tissue culture response of the species to improve the condition of the plant tissue to be transformed. Typically, a suitable tissue culture response has been obtained by optimizing medium components, explant material and source, and/or growing conditions. This has led to some success, but it still takes a significant amount of effort to efficiently obtain a sufficient number of independent transgenic events quickly. It would save considerable time and money if genes could be more efficiently introduced. Accordingly, methods are needed in the art to increase transformation efficiencies in a wide variety of plant species including those of the Setaria genus including S. viridis.

### SUMMARY OF THE INVENTION

The present invention provides an improved method for stably transforming S. viridis, which is a widely recognized model C4 grass. This model plant species can serve as a gene discovery/validation platform for maize, sugarcane, and other economically important crops. Improving the transformation efficiency of S. viridis is important because large numbers of transgenic plants are needed to enable studies on the effect of a large number of candidate genes or gene combinations within a given period of time. The method of the present invention is less labor-intensive than currently available protocols and provides improved transformation efficiency relative to previously developed transformation protocols for Setaria species. The method involves inducing callus growth from mature embryos at a light intensity of 5-30 μE m⁻² sec⁻¹, pre-treating quality callus on CSM medium for 3 to 5 days, growing Agrobacterium cells harboring a functional plant transformation vector for three days at a temperature of 19-22° C., re-suspending the Agrobacterium cells in infection medium to an optical density of less than 1.0 at 600 nm, co-cultivating the resuspended Agrobacterium cells with callus tissue in infection medium containing greater than 30 g/L sucrose, culturing the infected cells on selection medium at a temperature of 25-35° C. for 32-49 days to produce transformed tissue expressing the nucleic acid, and regenerating the transformed tissue on at least one regeneration medium to produce a transformed plant. Using the methods of the invention, transformation efficiencies of greater than about 10% up to greater than about 20% can be achieved.

Embodiments of the invention include:

1. A method of transforming callus derived from mature embryos of Setaria species comprising:
(a) inducing callus growth from mature embryos at a light intensity of 5-30 μE m⁻² sec⁻¹,
(b) pre-treating quality callus on CSM medium for 3 to 5 days,
(c) growing Agrobacterium cells harboring a functional plant transformation vector for three days at a temperature of 19-22° C.,
(d) re-suspending the Agrobacterium cells in infection medium to an optical density of less than 1.0 at 600 nm,
(e) co-cultivating the resuspended Agrobacterium cells with callus tissue in infection medium containing greater than 30 g/L sucrose,
(f) culturing the infected cells on selection medium at a temperature of 25-35° C. for 32-49 days to produce transformed tissue expressing the nucleic acid, and
(g) regenerating the transformed tissue on at least one regeneration medium to produce a transformed plant,

wherein the resulting transformation efficiency is at least 20%.

2. The method of embodiment 1 wherein said callus is derived from Setaria viridis.

3. The method of embodiment 1 wherein said callus is derived from Setaria italica.

4. The method of embodiment 1 wherein said inducing callus growth occurs at a light intensity of 15-25 μE m⁻² sec⁻¹.

5. The method of embodiment 1 wherein said inducing callus growth occurs at a light intensity of 10-20 μE m⁻² sec⁻¹.

6. The method of embodiment 1 wherein said Agrobacterium cells are resuspended in infection medium at an optical density of 0.10-0.24.

7. The method of embodiment 1 wherein said Agrobacterium cells are resuspended in infection medium at an optical density of 0.10-0.20.

8. The method of embodiment 1 wherein said Agrobacterium cells are resuspended in infection medium at an optical density of 0.12-0.18.

9. The method of embodiment 1 wherein said Agrobacterium cells are resuspended in infection medium at an optical density of 0.14-0.16.

10. The method of embodiment 1 wherein said Agrobacterium cells are resuspended in infection medium at an optical density of 0.15.

11. The method of embodiment 1 wherein said infected cells are cultured on selection medium at a temperature of 26-30° C.

12. The method of embodiment 1 wherein said infected cells are cultured on selection medium at a temperature of 28° C.

13. The method of embodiment 1 wherein said growing Agrobacterium cells occurs on solid YEP medium containing the appropriate antibiotics for plasmid maintenance.

14. The method of embodiment 2 wherein said callus derived from Setaria viridis is derived from the accession A10.1.

15. The method of embodiment 2 wherein said callus derived from Setaria viridis is derived from the accession ME034V.

16. The method of embodiment 1 wherein said CSM medium does not contain a cytokinin.

17. The method of embodiment 1 wherein said selection medium is CSM supplemented with appropriate chemicals to affect selection.

18. The method of embodiment 1 wherein said selection medium is CIM supplemented with appropriate chemicals to affect selection.

19. The method of embodiment 17 or embodiment 18 wherein said appropriate chemicals to affect selection are selected from the group of hygromycin, bialaphos, and kanamycin.

20. The method of embodiment 1 wherein said co-cultivating the resuspended Agrobacterium cells occurs in the dark for three days.

21. The method of embodiment 1 wherein said culturing the infected cells on selection medium occurs in the dark.

### DETAILED DESCRIPTION OF THE INVENTION

Improved methods for transformation and regeneration of Setaria species are provided herein. The examples below detail the application of these methods. These improved methods result in significantly increased plant transformation frequency as compared to previously established transformation protocols.

An “increased transformation efficiency,” as used herein, refers to any improvement, such as an increase in transformation frequency and quality of events that impact the overall efficiency of the transformation process by reducing the amount of resources required. “Transformation efficiency” as used herein is calculated by dividing the number of regenerated plants containing resulting from a given transformation experiment and containing the DNA of interest by the number of callus pieces used for said transformation experiment. The methods of the invention are able to increase transformation efficiency greater than about 10%, greater than about 15%, and greater than about 20% as compared to art recognized methods for transformation of Setaria.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella et al. (1983) EMBO J. 2:987-992); methotrexate (Herrera Estrella et al. (1983) Nature 303:209-213; Meijer et al. (1991) Plant Mol. Biol. 16:807-820); hygromycin (Waldron et al. (1985) Plant Mol. Biol. 5:103-108; Zhijian et al. (1995) Plant Science 108:219-227); streptomycin (Jones et al. (1987) Mol. Gen. Genet. 210:86-91); spectinomycin (Bretagne-Sagnard et al. (1996) Transgenic Res. 5:131-137); bleomycin (Hille et al. (1990) Plant Mol. Biol. 7:171-176); sulfonamide (Guerineau et al. (1990) Plant Mol. Bio. 15:127-136); bromoxynil (Stalker et al. (1988) Science 242:419-423); glyphosate (Shaw et al. (1986) Science 233:478-481); phosphinothricin (DeBlock et al. (1987) EMBO J. 6:2513-2518).

Although Setaria viridis has been proposed as an excellent model plant species for studying traits of potential agronomic performance, genetic transformation of Setaria species has historically been difficult to perform with a high efficiency. Few reports of Setaria transformation exist in the scientific literature. The first S. viridis transformation protocol that we are aware of was made public in 2010 (Brutnell et al (2010) Plant Cell 22:2537-2544); a transformation efficiency was not reported in this publication. The laboratory of Joyce Van Eck has also worked to optimize the Setaria transformation protocol (van Eck and Swartwood (2014) The First Annual Setaria Genetics Conference Abstracts. Beijing; Swartwood and van Eck (2014) The First Annual Setaria Genetics Conference Abstracts. Beijing; van Eck and Swartwood (2015) Methods Mol Biol 1223:57-67); 5-10% transformation efficiencies are reported for S. viridis transformation using these protocols. A recent publication reported efficiencies of up to 29% for transformation of S. viridis, but this efficiency was obtained only in one experiment; the overall efficiency obtained by this group was 13.8% (Martins et al 2015 Biotechnology Reports 6:41-44).

An increased “transformation efficiency,” as used herein, refers to any improvement, such as an increase in transformation frequency and quality of events that impact the overall efficiency of the transformation process by reducing the amount of resources required. Transformation efficiency can be calculated by dividing the number of transgenic plants recovered from a given transformation experiment by the number of callus pieces used for said transformation experiment. In order to provide reliable and reproducible transformation efficiencies, such efficiencies should be calculated from at least one hundred (100) callus pieces. The use of too few callus pieces may result in an overestimate or underestimate of the transformation efficiency that may be achieved by a given transformation protocol.

The transformation protocols and methods of the present invention provide a transformation efficiency of at least 20%. This is an increased efficiency over the previously published methods of Setaria transformation.

The following examples are offered by way of illustration and not by way of limitation.

### EXPERIMENTAL

### Example 1

### Media Compositions

### YEP Medium:

5 g/L yeast extract, 10 g/L peptone, 5 g/L NaCl, 15 g/L Bacto-agar. Adjust pH to 6.8 with NaOH. Appropriate antibiotics (Kanamycin stock at 50 mg/L) should be added to the medium when cooled to 50° C. after autoclaving.
S. viridis Callus Induction Medium (CIM):
4.33 g/L MS salt and MS vitamins, 40 g/L maltose, 35 mg/L ZnSO₄.7H₂O, 0.6 mg/L CuSO₄.5H2O, 2.0 mg/L 2,4-D (1 mg/mL), 8.0 g/L agar. Adjust with KOH to pH 5.8, autoclave. Filter-sterilized 0.5 mg/L Kinetin is added prior to use.
S. viridis Callus Subculture Medium (CSM):
4.33 g/L MS salt and MS vitamins, 40 g/L maltose, 35 mg/L ZnSO₄.7H₂O, 0.6 mg/L CuSO₄.5H₂O, 2.0 mg/L 2, 4-D (1 mg/mL), 8.0 g/L agar. Adjust with KOH to pH 5.8, autoclave.

### Co-Cultivation Medium:

4.33 g/L MS salt and MS vitamins, 30 g/L sucrose, 2.5 mL/L 2,4-D (1 mg/mL), 8.0 g/L agar. Adjust with KOH to pH 5.8, autoclave. Add 1 mL/L acetosyringone (100 mM) before use.

### Infection Medium:

2.16 g/L MS salt, 1 mL/L MS vitamins (1000X), 68.5 g/L sucrose, 36 g/L glucose, 0.115 g/L L-proline, 1.5mL/L 2,4-D (1 mg/mL). Adjust with KOH to pH 5.2, autoclave. Add 1 mL/L acetosyringone (100 mM) before use.

### Selection Medium:

4.33 g/L MS salt and MS vitamins, 40 g/L maltose, 35 mg/L ZnSO4.7H2O, 0.6 mg/L CuSO₄.5H₂O, 2 mg/mL 2,4-D, 8.0 g/L Agar. Adjust with KOH to pH 5.8, autoclave. Filter-sterilized 40 mg/L hygromycin, 100 mg/L Timentin, 150 mg/L cefotaxime cocktail with or without kinetin is added prior to use.

### Regeneration Medium I

4.33 g/L MS salt and vitamins, 30 g/L sucrose, adjusted with KOH to pH 5.8, autoclave. Filter-sterilized 0.2 mg/L Kinetin, 20 mg/L hygromycin, 100 mg/L Timentin, 150 mg/L cefotaxime cocktail is added prior to use.

### Regeneration Medium II:

2.16 g/L MS Salts and vitamins, 30 g/L sucrose, 2.6 g/L Phytogel (pH 5.8).

### Example 2

S. viridis A10.1 transformation

### Materials:

Plant materials: Compact light-yellow colored S. viridis calli derived from S. viridis cultivar A10.1

Agrobacterium strain: AGL-1 or LBA4404 harboring binary vector pMDC99 or super binary vector pSB1 with a strong constitutive promoter driving an appropriate selectable marker gene (such as Hpt or Bar/PAT).

### Transformation:

1. Transfer compact calli derived from mature embryos and grown in dim light (10-20 μE m⁻² s⁻¹) to CSM medium at 28° C. for three to five days.

2. Agrobacterium cultures (AGL-1 hosting regular binary vector) are grown for three days at 19 to 22° C. on solid YEP medium amended with 50 mg/L kanamycin.

3. A small amount of bacterial culture is scraped from the plate and suspended in approximately 15 mL of liquid Infection Medium in a 50 mL conical tube. Adjust the optical density to OD_{600=0.15} before use.

4. For each construct, transfer a small amount of actively growing calli to a tube. Using sterile forceps, subculture compact calli from their original plates and transfer them to their corresponding petri dish. Callus pieces should be approximately 2-4 mm in diameter, as if they are too small, they will not survive the transformation.

5. Add 4 mL Agrobacterium suspension, vortex at full speed for 15 seconds, then allow calli to incubate in culture at room temperature for 5-7 minutes in the dark.

6. Place infected calli onto dry filter paper in a 100×15 mm plate and leave in hood until no major trace of liquid is visible.

7. Transfer calli with filter paper to co-cultivation plate, re-arrange the calli to ensure no aggregation.

8. Co-cultivation plates are incubated in the dark at 25° C. for three days.

9. Transfer infected calli off the filter paper and place on top of Selection Medium.

10. Selection plates are wrapped and placed in the dark at 28° C.

11. Every two weeks, the tissue is sub-cultured onto fresh Selection Medium. There will be a five to six week selection period with three separate sub-cultures to fresh Selection Medium.

12. Transfer active growing calli/emerging shoots to regeneration/selection plates containing Regeneration Medium I for shoot induction at 28° C. in light growth chamber until shoots become excisable (in about 2 weeks).

13. Transfer all regenerated shoots with forceps and Regeneration Medium II for rooting/selection at 28° C. and 16/8 photoperiods.

Transformations were performed according to the protocols described above. Following the transfer of regenerated shoots to Regeneration Medium II and allowing sufficient time for the plants to grow in this medium, tissue samples were collected and DNA was extracted from these tissue samples. A PCR-based assay was performed to detect the presence of the selectable marker gene (i.e., the gene encoding a protein that provides antibiotic or herbicide resistance for selection). Transformation efficiencies were calculated by dividing the number of PCR-positive rooted plantlets by the number of callus pieces that were used for the transformation experiment. Twenty-one transformation experiments were performed with vectors containing a selectable marker gene as well as different genes of interest, with the resulting transformation efficiencies shown in Table 1.

TABLE 1
S. viridis accession A10.1 transformation efficiencies

| | Callus Pieces | | |
|---|---|---|---|
| | Used | PCR+ | Efficiency |
| | 40 | 12 | 30.0% |
| | 40 | 2 | 5.0% |
| | 40 | 3 | 7.5% |
| | 40 | 1 | 2.5% |
| | 40 | 7 | 17.5% |
| | 40 | 7 | 17.5% |
| | 40 | 11 | 27.5% |
| | 40 | 10 | 25.0% |
| | 40 | 8 | 20.0% |
| | 40 | 7 | 17.5% |
| | 40 | 12 | 30.0% |
| | 40 | 13 | 32.5% |
| | 40 | 6 | 15.0% |
| | 20 | 3 | 15.0% |
| | 20 | 14 | 70.0% |
| | 20 | 10 | 50.0% |
| | 20 | 12 | 60.0% |
| | 20 | 14 | 70.0% |
| | 20 | 10 | 50.0% |
| | 20 | 21 | 105.0% |
| | 20 | 19 | 95.0% |
| Totals: | 680 | 202 | 29.7% |

### Example 3

S. viridis ME034V transformation

### Materials:

Plant materials: Compact light-yellow colored S. viridis calli derived from S. viridis cultivar ME034V

Agrobacterium strain: AGL-1 or LBA4404 harboring binary vector pMDC99 or super binary vector pSB1 with a strong constitutive promoter driving an appropriate selectable marker gene (such as Hpt or Bar/PAT).

### Transformation:

1. Transfer compact calli derived from mature embryos and grown in dim light (10-20 μE m⁻² s⁻¹) to CIM medium at 28° C. for three to five days.

2. Agrobacterium cultures (AGL-1 hosting regular binary vector) are grown for three days at 19 to 22° C. on solid YEP medium amended with 50 mg/L kanamycin.

3. A small amount of bacterial culture is scraped from the plate and suspended in approximately 15 mL of liquid Infection Medium in a 50 mL conical tube. Adjust the optical density to OD_{600=0.15} before use.

4. For each construct, transfer a small amount of actively growing calli to a tube. Using sterile forceps, subculture compact calli from their original plates and transfer them to their corresponding petri dish. Callus pieces should be approximately 2-4 mm in diameter, as if they are too small, they will not survive the transformation.

5. Add 4 mL Agrobacterium suspension, vortex at full speed for 15 seconds, then allow calli to incubate in culture at room temperature for 5-7 minutes in the dark.

6. Place infected calli onto dry filter paper in a 100×15 mm plate and leave in hood until no major trace of liquid is visible.

7. Transfer calli with filter paper to co-cultivation plate, re-arrange the calli to ensure no aggregation.

8. Co-cultivation plates are incubated in the dark at 25° C. for three days.

9. Transfer infected calli off the filter paper and place on top of Selection Medium.

10. Selection plates are wrapped and placed in the dark at 28° C.

11. Two weeks after the initial transfer to Selection Medium, the tissue is sub-cultured onto fresh Selection Medium. Two weeks after this sub-culture, the tissue is transferred to a fresh plate containing CIM medium supplemented with 40-60 mg/L hygromycin.

12. Transfer active growing calli/emerging shoots to regeneration /selection plates containing Regeneration Medium I for shoot induction at 28° C. in light growth chamber until shoots become excisable (in about 2 weeks).

13. Transfer all regenerated shoots with forceps and Regeneration Medium II for rooting/selection at 28° C. and 16/8 photoperiods.

Transformations were performed according to the protocols described above. Following the transfer of regenerated shoots to Regeneration Medium II and allowing sufficient time for the plants to grow in this medium, tissue samples were collected and DNA was extracted from these tissue samples. A PCR-based assay was performed to detect the presence of the selectable marker gene (i.e., the gene encoding a protein that provides antibiotic or herbicide resistance for selection). Transformation efficiencies were calculated by dividing the number of PCR-positive rooted plantlets by the number of callus pieces that were used for the transformation experiment. Eight transformation experiments were performed with the resulting transformation efficiencies shown in Table 2.

TABLE 2
S. viridis accession ME034V transformation efficiencies

| | Callus Pieces | | |
|---|---|---|---|
| | Used | PCR+ | Efficiency |
| | 27 | 21 | 77.8% |
| | 45 | 60 | 133.3% |
| | 20 | 9 | 45.0% |
| | 20 | 17 | 85.0% |
| | 20 | 2 | 10.0% |
| | 20 | 29 | 145.5% |
| | 20 | 15 | 75.0% |
| | 20 | 31 | 155.0% |
| | 20 | 18 | 90.0% |
| Totals: | 212 | 202 | 95.3% |

1. A method of transforming callus derived from mature embryos of Setaria species comprising:
(a) inducing callus growth from mature embryos at a light intensity of 5-30 μE m⁻² sec⁻¹,
(b) pre-treating quality callus on CSM medium for 3 to 5 days,
(c) growing Agrobacterium cells harboring a functional plant transformation vector for three days at a temperature of 19-22° C.,
(d) re-suspending the Agrobacterium cells in infection medium to an optical density of less than 1.0 at 600 nm,
(e) co-cultivating the resuspended Agrobacterium cells with callus tissue in infection medium containing greater than 30 g/L sucrose,
(f) culturing the infected cells on selection medium at a temperature of 25-35° C. for 32-49 days to produce transformed tissue expressing the nucleic acid, and
(g) regenerating the transformed tissue on at least one regeneration medium to produce a transformed plant,
wherein the resulting transformation efficiency is at least 20%.

2. The method of claim 1 wherein said callus is derived from Setaria viridis.

3. The method of claim 1 wherein said callus is derived from Setaria italica.

4. The method of claim 1 wherein said inducing callus growth occurs at a light intensity of 15-25 μE m⁻² sec^{—1}.

5. The method of claim 1 wherein said inducing callus growth occurs at a light intensity of 10-20 μE m ² sec ¹.

6. The method of claim 1 wherein said Agrobacterium cells are resuspended in infection medium at an optical density of 0.10-0.24.

7. (canceled)

8. The method of claim 1 wherein said Agrobacterium cells are resuspended in infection medium at an optical density of 0.12-0.18.

9. The method of claim 1 wherein said Agrobacterium cells are resuspended in infection medium at an optical density of 0.14-0.16.

10. The method of claim 1 wherein said Agrobacterium cells are resuspended in infection medium at an optical density of 0.15.

11. The method of claim 1 wherein said infected cells are cultured on selection medium at a temperature of 26-30° C.

12. The method of claim 1 wherein said infected cells are cultured on selection medium at a temperature of 28° C.

13. The method of claim 1 wherein said growing Agrobacterium cells occurs on solid YEP medium containing the appropriate antibiotics for plasmid maintenance.

14. The method of claim 2 wherein said callus derived from Setaria viridis is derived from the accession A10.1.

15. The method of claim 2 wherein said callus derived from Setaria viridis is derived from the accession ME034V.

16. The method of claim 1 wherein said CSM medium does not contain a cytokinin.

17. The method of claim 1 wherein said selection medium is CSM supplemented with appropriate chemicals to affect selection.

18. The method of claim 1 wherein said selection medium is CIM supplemented with appropriate chemicals to affect selection.

19. The method of claim 17 wherein said appropriate chemicals to affect selection are selected from the group of hygromycin, bialaphos, and kanamycin.

20. The method of claim 1 wherein said co-cultivating the resuspended Agrobacterium cells occurs in the dark for three days.

21. The method of claim 1 wherein said culturing the infected cells on selection medium occurs in the dark.
